(19) United States
(12) Patent Application Publication
(10) Pub. No.: US 2025/0002898 A1
(43) Pub. Date: Jan. 2, 2025

(54) DEVICES, SYSTEMS AND METHODS FOR PROCESSING

(71) Applicant: Flagship Pioneering Innovations VI, LLC, Cambridge, MA (US)

(72) Inventors: Tushar Kanti MISRA, Norfolk, MA (US); Olga SELTSER, Burlington, MA (US); Daniel P. CINICOLA, Sudbury, MA (US)

(21) Appl. No.: 18/291,620

(22) PCT Filed: Jul. 27, 2022
(86) PCT No.: PCT/US2022/038427

§ 371 Date: Jan. 24, 2024

Related U.S. Application Data
(60) Provisional application No. 63/203,638, filed on Jul. 27, 2021.

(51) Int. Cl. C12N 15/10

(52) U.S. Cl. CPC C12N 15/1017

(57) ABSTRACT

This invention relates generally to devices, systems, and methods for the preparation and processing of circular polyribonucleotides comprising the use of a pressurization device comprising a reservoir comprising (a) a filtrate chamber and a (b) waste collection chamber separated by (c) a filter; the reservoir further comprising (d) a removable cap in contact with a pressure inlet port; wherein the device is in fluidic communication with a positive pressure source.

### BACKGROUND

Methods are needed for the production and processing of circular polyribonucleotides.

### SUMMARY

This invention relates generally to devices, systems, and methods for the preparation and processing of circular polyribonucleotides.

In one aspect, the invention features a method of processing circular polyribonucleotides. This method includes the steps of: (a) providing a device that includes a reservoir that has (i) a filter disposed between (ii) a filtrate chamber and a (iii) waste collection chamber the reservoir further including a removable cap having a pressure inlet port; wherein the device is in fluidic communication with a positive pressure source through the pressure inlet port; (b) providing circular polyribonucleotides in a first fluid in the filtrate chamber; (c) pressurizing the reservoir with the positive pressure source such that the first fluid passes through the filter and the circular polyribonucleotides do not pass through the filter; (d) applying a second fluid to the reservoir; and (e) pressurizing the reservoir with the positive pressure source such that the second buffer passes through the filter and the circular polyribonucleotides do not pass through the filter.

In some embodiments, the method further includes the step of repeating steps (d) and (e) from 1 to 5 times. In some embodiments, the method further includes the step of (f) aspirating the circular polynucleotides from the filter following step (e). In some embodiments, from 1 mg to 1000 mg of circular polyribonucleotides are aspirated from the filter. In some embodiments, from 50 mg to 150 mg of circular polyribonucleotides are aspirated from the filter. In some embodiments, about 100 mg of circular polyribonucleotides are aspirated from the filter.

In some embodiments, the second fluid is applied through a fluid inlet port in the cap. In some embodiments, the fluid inlet port is in fluidic communication with a fluid source. In some embodiments, the first and/or second fluid is a liquid. In some embodiments, the liquid is a solution. In some embodiments, the solution is a buffer. In some embodiments, the buffer is a formulation buffer, storage buffer, or purification buffer.

In some embodiments, pressurizing step (c) and step (d) include pressurizing the reservoir with from 10 PSI to 100 PSI. In some embodiments, applying step (d) includes applying from 5 mL to 15 mL of fluid to the reservoir.

In another aspect, the invention features a device includes: a reservoir that has a filtrate chamber and a waste collection chamber separated by a filter, and a removable cap in contact with a pressure inlet port; wherein the device is in fluidic communication with a positive pressure source.

In some embodiments, the reservoir is a tube. In some embodiments, the tube is a centrifugal tube. In some embodiments, the filtrate chamber is funneled. In some embodiments, the cap further includes a fluid inlet port. In some embodiments, the volume of the reservoir is from 5 mL to 100 mL. In some embodiments, the volume of the filtrate chamber is from 5 mL to 15 mL. In some embodiments, the filter is from 2 kDa to 200 kDa. In some embodiments, the filtrate chamber and filter are stacked on, screwed into, or nested in the reservoir. In some embodiments, the pressure inlet port is sealed.

In some embodiments, the device further includes a sensor (e.g., a liquid detection sensor). In some embodiments, the liquid detection sensor is selected from the list consisting of an optical sensor, a vibrating sensor, an ultrasonic sensor, a float sensor, a capacitance sensor, a radar sensor, a conductivity sensor, or a resistance sensor. In some embodiments, the sensor is an optical sensor.

In another aspect, the invention features a system that includes (i) a filter tray having a sample reservoir including filtrate chamber having a filter, the filter tray disposed between (ii) a lid having a pressure inlet port, and (iii) a waste reservoir; wherein the pressure inlet port is in fluidic communication with a (iv) a positive pressure source.

In some embodiments, the filtrate chamber is funneled. In some embodiments, the volume of the filtrate chamber is from 5 mL to 15 mL. In some embodiments, the waste reservoir includes a gravity or vacuum drain. In some embodiments, the system further includes a liquid detection sensor. In some embodiments, the system further includes a vent. In some embodiments, the filter tray, lid, and waste reservoir are sealably connected together. In some embodiments, the filtrate chamber is stacked on, screwed into, or nested in the sample reservoir. In some embodiments, the lid further includes a fluid inlet port. In some embodiments, the filter tray includes a plurality of sample reservoirs.

### Definitions

The present invention will be described with respect to particular embodiments and with reference to certain figures, but the invention is not limited thereto but only by the claims. Terms as set forth hereinafter are generally to be understood in their common sense unless indicated otherwise.

As used herein, the term “circular polyribonucleotide” or “circRNA” or “circular RNA” are used interchangeably and mean a polyribonucleotide molecule that has a structure having no free ends (i.e., no free 3′ and/or 5′ ends), for example a polyribonucleotide molecule that forms a circular or end-less structure through covalent or non-covalent bonds. In some embodiments, a circular polyribonucleotide is a “covalently closed polyribonucleotide” that formed a circular or end-less structure through covalent bonds.

As used herein, the terms “circular polyribonucleotide sample”, “circRNA sample”, and “circular RNA sample” are used interchangeably and mean a composition including circRNA molecules and a solution.

As used herein, the term “expression sequence” refers to a nucleic acid sequence that encodes a product, e.g., a peptide or polypeptide, or a regulatory nucleic acid.

“Polypeptide” and “protein” are used interchangeably and refer to a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics).

As used herein, the term “aptamer sequence” or “aptamer” refers to a non-naturally occurring or synthetic oligonucleotide that specifically binds to a target molecule. Typically, an aptamer is from 20 to 250 nucleotides. Typically, an aptamer binds to its target through secondary structure rather than sequence homology.

As used herein, the term “binding site” refers to a region of the circular polyribonucleotide that interacts with another entity, e.g., a chemical compound, a protein, a nucleic acid, etc. A binding site can include an aptamer sequence.

As used herein, a “buffer” is a solution that can resist pH change upon the addition of an acidic or basic components. Examples of “buffers” include “formulation buffers” for use in the formulation of a composition, “storage buffers” for use in storage of a composition, “purification buffers” for use in purification, and the like.

The term “buffer exchange”, as used herein, refers to process of removing a molecule or particle, such as polyribonucleotides, from a first buffer, for incorporation into a second buffer.

The term “fluid communication,” as used herein, refers to a connection between at least two device elements, e.g., a reservoir, pressure source, etc., that allows for fluid to move between such device elements with or without passing through one or more intervening device elements.

As used herein, a “solution” is a homogenous mixture of two or more substances, including a solvent and a solute.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Circular polyribonucleotides are described, e.g., in U.S. Patent Publication No. US2020/0306286, and in PCT Patent Publication Nos. WO2020/181013 and WO2020/023655, which are incorporated herein by reference.

### BRIEF DESCRIPTION OF THE DRAWINGS

The following detailed description of the embodiments of the invention will be better understood when read in conjunction with the appended drawings. For the purpose of illustrating the invention, there are shown in the drawing embodiments, which are presently exemplified. It should be understood, however, that the invention is not limited to the precise arrangement and instrumentalities of the embodiments shown in the drawings.

FIG. 1 shows pressurized device including a reservoir having a filter disposed between a filtrate chamber and a waste collection chamber, as well as a cap having a pressure inlet port, wherein the pressurized device is under positive pressure, and the pressure is exerted through the positive pressure port.

FIG. 2 shows a pressurized device including a reservoir having a filter disposed between a filtrate chamber and a waste collection chamber, as well as a cap having both pressure inlet port and a fluid inlet port, wherein the pressurized device is under positive pressure, and the pressure is exerted through the pressure inlet port; and wherein fresh fluid may be applied to the filtrate chamber through the fluid inlet port.

FIGS. 3A-3C show a pressurized system or pressurized robot assembly including a lid having at least one port, a filter tray having a plurality of sample reservoirs having filters, and a waste reservoir.

### DETAILED DESCRIPTION

The invention relates generally to devices, systems, and methods for the processing of circular polyribonucleotide samples, e.g., for buffer exchange of such samples. The inventors have found that the pressurization of fluids, such as solutions (e.g., buffers), including circular polynucleotides leads to a simplified and hastened liquid exchange. One embodiment of the present invention is a device configured to transfer circular polyribonucleotides from a first fluid to a second fluid using pressurization, e.g., a combination of pressurization and filtration.

In some embodiments, circular polyribonucleotides are polyribonucleotides that are circular, i.e., polyribonucleotides that have no free ends. Circular polyribonucleotides are described, e.g., in U.S. Patent Publication No. US2020/0306286, and in PCT Patent Publication Nos. WO2020/181013 and WO2020/023655, which are incorporated herein by reference. Circular polyribonucleotides are also described in, e.g., WO2015/034925, WO2016/011222, U.S. Ser. No. 10/407,683, WO2017/222911, WO2021/041541, WO2019/236673, WO2020/237227, WO2016/197121, WO2018/191722, and WO2020/023595, which are incorporated herein by reference. In some embodiments, circular polyribonucleotides have improved stability, increased half-life, reduced immunogenicity, and/or improved functionality (e.g., of a function described herein) compared to a corresponding linear polyribonucleotides.

In some embodiments, the circular polyribonucleotides are at least about 20 base pairs, at least about 30 base pairs, at least about 40 base pairs, at least about 50 base pairs, at least about 75 base pairs, at least about 100 base pairs, at least about 200 base pairs, at least about 300 base pairs, at least about 400 base pairs, at least about 500 base pairs, or at least about 1,000 base pairs.

In some embodiments, the circular polyribonucleotides have lengths of at least 100 nucleotides, at least 200 nucleotides, at least 300 nucleotides, at least 400 nucleotides, at least 500 nucleotides, at least 1,000 nucleotides, at least 2,000 nucleotides, at least 3,000 nucleotides, at least 4,000 nucleotides, at least 5,000 nucleotides, at least 7,500 nucleotides, at least 10,000 nucleotides, at least 15,000 nucleotides, at least 20,000 nucleotides may be useful.

In some embodiments, the circular polyribonucleotide includes at least one expression sequence, e.g., encoding a polypeptide. In some embodiments, the expression sequence encodes a peptide or polynucleotide. In some embodiments, the circular polyribonucleotide includes a plurality of expression sequences, either the same or different. In some embodiments, the expression sequence has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the expression sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

In some embodiments, the circular polyribonucleotide includes one or more elements, such as expression sequences, and those one or more elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, or about 1000 nucleotides. In some embodiments, one or more elements are contiguous with one another, e.g., lacking a spacer element. In one embodiment, the ratio of the spacer sequence to a non-spacer sequence of the circular polyribonucleotide, e.g., expression sequences, of about 0.05:1, about 0.06:1, about 0.07:1, about 0.08:1, about 0.09:1, about 0.1:1, about 0.12:1, about 0.125:1, about 0.15:1, about 0.175:1, about 0.2:1, about 0.225:1, about 0.25:1, about 0.3:1, about 0.35:1, about 0.4:1, about 0.45:1, about 0.5:1, about 0.55:1, about 0.6:1, about 0.65:1, about 0.7:1, about 0.75:1, about 0.8:1, about 0.85:1, about 0.9:1, about 0.95:1, about 0.98:1, about 1:1, about 1.02:1, about 1.05:1, about 1.1:1, about 1.15:1, about 1.2:1, about 1.25:1, about 1.3:1, about 1.35:1, about 1.4:1, about 1.45:1, about 1.5:1, about 1.55:1, about 1.6:1, about 1.65:1, about 1.7:1, about 1.75:1, about 1.8:1, about 1.85:1, about 1.9:1, about 1.95:1, about 1.975:1, about 1.98:1, or about 2:1. In one embodiment, the spacer sequence is configured to provide conformational flexibility between elements of the circular polyribonucleotide on both sides of the spacer sequence.

In some embodiments, the circular polyribonucleotides may include one or more repetitive elements. In some embodiments, the circular polyribonucleotides include one more more modifications. In some embodiments, the circular polyribonucleotides include at least 10%, 20%, 30%, 40%, 50%, 60%, 70%, or 80% modified nucleotides.

A circular polyribonucleotide can include at least one binding site for a target, e.g., for a binding moiety of a target. A circular polyribonucleotide can include at least one aptamer sequence that binds to a target. In some embodiments, the circular polyribonucleotide includes one or more binding sites for one or more targets. Targets include, but are not limited to, nucleic acids (e.g., RNAs, DNAs, RNA-DNA hybrids), small molecules (e.g., drugs, fluorophores, metabolites), aptamers, polypeptides, proteins, lipids, carbohydrates, antibodies, viruses, virus particles, membranes, multi-component complexes, organelles, cells, other cellular moieties, any fragments thereof, and any combination thereof. In some embodiments, circular polyribonucleotides include a binding site to a single target or a plurality of (e.g., two or more) targets. In one embodiment, a single circular polyribonucleotides includes 2, 3, 4, 5, 6, 7, 8, 9, 10, or more different binding sites for a single target. In one embodiment, a single circular polyribonucleotide includes 2, 3, 4, 5, 6, 7, 8, 9, 10, or more of the same binding sites for a single target. In one embodiment, a single circular polyribonucleotide includes 2, 3, 4, 5, 6, 7, 8, 9, 10, or more different binding sites for one or more different targets. In one embodiment, two or more targets are in a sample, such as a mixture or library of targets, and the sample includes circular polyribonucleotides including two or more binding sites that bind to the two or more targets.

In the processing of circular ribonucleotide samples, it may be necessary to transfer circular ribonucleotides from a first fluid to a second fluid, wherein the second fluid is more amenable for further processing, formulation, storage, purification, etc. The present invention fluid exchange may be done without emptying the reservoir including the filter having circular polyribonucleotides, providing a more hands off, robust methodology. In one embodiment, the process may be further enhanced with fluid detection sensors to provide a controlled process to avoid over-drying the filter itself. Circular polyribonucleotide samples may be distributed in fluids. In one embodiment, the fluid is a liquid or gas. In one embodiment, the fluid is a solution. In one embodiment, the solution is a buffer. Buffers to be used in the present invention include any aqueous solution that maintains the circular polyribonucleotide at a stable pH and/or stable structure. Buffers may include formulation buffers for the use in the formulation of a composition, storage buffers for the use in storage of a composition, and purification buffers for the use of purification, and the like. Buffers may include sodium citrate buffers, phosphate-buffered saline (PBS), Tris-based buffers, sodium phosphate, water, and the like. The present invention may be used with a plurality of fluids in series (e.g., used one after another) or parallel (e.g., used together).

In some embodiments, off the shelf filters may be used with the present devices and systems.

Filters include the Amicon® Ultra Filters, Pierce™ Protein Concentrator PES Filters, etc. A variety of filter pore sizes are imagined, including, but not limited to, <2 kDa, 3 kDa, 20 kDa, 30 kDa, 50 kDa, 100 kDa, 200 kDa, and the like. Filters having a variety of molecular weight cut off (MWCO) are imagined, including, but not limited to, 10 MWCO, 30 MWCO, and 100 MWCO.

A variety of circular polyribonucleotide concentrations are imagined, including, but not limited to 0.5-2.0 mg/mL.

A variety of resulting circular polyribonucleotide weights for incorporation into a second fluid, such as a buffer, are imagined, including, 1-200 mg, e.g., 1 mg, 2 mg, 3 mg, 4 mg, 5 mg, 10 mg, 20 mg, 30 mg, 40 mg, 50 mg, 60 mg, 70 mg, 80 mg, 90 mg, 100 mg, 150 mg, or 200 mg.

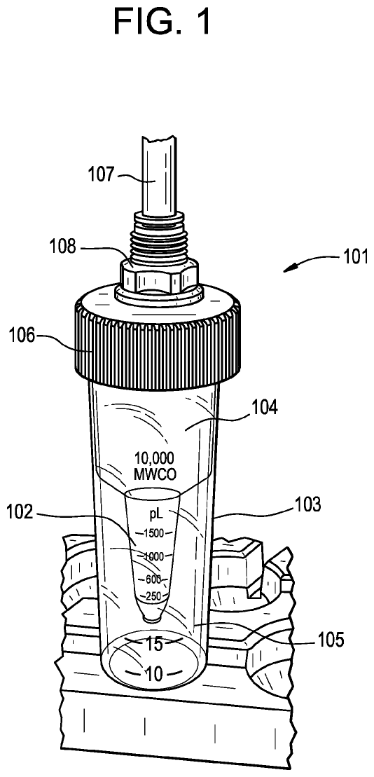

FIG. 1 shows pressurized device (101) including a reservoir (103) having a filter (102) disposed between a filtrate chamber (104) and a waste collection chamber (105), as well as a cap (106) having a pressure inlet port (108), wherein the pressurized device is under positive pressure, and the pressure is exerted through the pressure inlet port (108). The pressurized device (101) may further include a fluidic connection component (107), which may place the reservoir in fluidic communication with a pressure source.

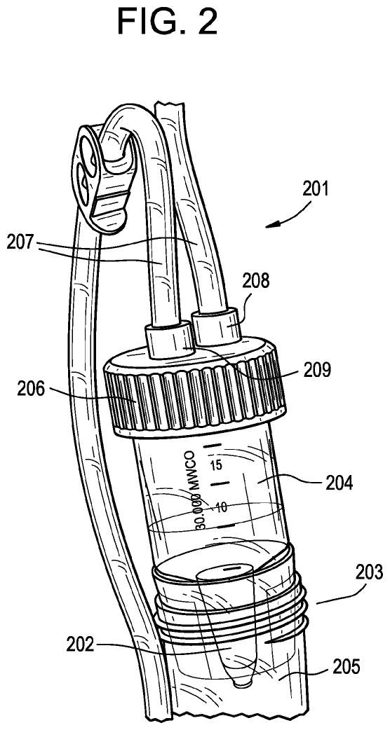

FIG. 2 shows an alternate embodiment of a pressurized device (201) including a reservoir (203) having a filter (202) disposed between a filtrate chamber (204) and a waste collection chamber (205), as well as a cap (206) having a pressure inlet port (208) and a fluid inlet port (209), wherein the pressurized device (201) is under positive pressure, and the pressure is exerted through the pressure inlet port (208); and wherein fresh fluid may be applied to the filtrate chamber (204) through the fluid inlet port (209). The pressurized device (201) may further include a fluidic connection component (207), which may place the reservoir in fluid communication with a pressure source. In some embodiments, the cap includes pressure inlet ports. The pressure inlet port may be connected to a pressure source by a fluidic connection component. In certain devices, methods, and systems described herein, positive pressure may be exerted by a positive pressure source via the pressure inlet port to force fluid through the filter. A range of pressures are envisaged for fluid exchange of circular polyribonucleotides, including from 10 PSI to 100 PSI, e.g., from 10 PSI to 20 PSI, 10 PSI to 25 PSI, 10 PSI to 30 PSI, 10 PSI to 40 PSI, 10 PSI to 50 PSI, 10 PSI to 60 PSI, 10 PSI to 70 PSI, 10 PSI to 80 PSI, 10 PSI to 90 PSI, 20 PSI to 25 PSI, 20 PSI to 30 PSI, 20 PSI to 40 PSI, 20 PSI to 50 PSI, 20 PSI to 60 PSI, 20 PSI to 70 PSI, 20 PSI to 75 PSI, 20 PSI to 80 PSI, 20 PSI to 90 PSI, 20 PSI to 100 PSI, 30 PSI to 40 PSI, 30 PSI to 50 PSI, 30 PSI to 60 PSI, 30 PSI to 70 PSI, 30 PSI to 75 PSI, 30 PSI to 80 PSI, 30 PSI to 90 PSI, 30 PSI to 100 PSI, 40 PSI to 50 PSI, 40 PSI to 60 PSI, 40 PSI to 70 PSI, 40 PSI to 75 PSI, 40 PSI to 80 PSI, 40 PSI to 90 PSI, 40 PSI to 100 PSI, 50 PSI to 60 PSI, 50 PSI to 70 PSI, 50 PSI to 75 PSI, 50 PSI to 80 PSI, 50 PSI to 90 PSI, 50 PSI to 100 PSI, 60 PSI to 70 PSI, 60 to 75 PSI, 60 PSI to 80 PSI, 60 PSI to 90 PSI, 60 PSI to 100 PSI, 70 PSI to 75 PSI, 70 PSI to 80 PSI, 70 PSI to 90 PSI, 70 PSI to 100 PSI, 80 PSI to 90 PSI, 80 PSI to 100 PSI, 90 PSI to 100 PSI, or about 10, 20, 30, 40, 50, 60, 65, 70, 80, 90, or 100 PSI. In one embodiment, the cap having the pressure inlet port(s) and/or the fluid inlet port(s) may be sealed in order to withstand pressurization. In one embodiment, the present reservoir, filter, cap, and related fluidic connections are connected together, e.g., clamped, in order to withstand pressurization. In one embodiment, metallic clamps may be used to clamp together the present device and system.

The positive pressure may be maintained over the reservoir for a period of time. The reservoir may be pressurized from one minute to several hours. In one embodiment, the reservoir is pressurized for less than 3 hours, more preferably for less than an hour. In one embodiment, the reservoir is pressurized for less than 10 minutes for initial fluid washes and pressurized from 10 minutes to 55 minutes for ensuing fluid washes. A range of pressurized filtration times are imagined, including for less than 5 minutes, 10 minutes, 15 minutes, 20 minutes, 30 minutes, 40 minutes, 45 minutes, an hour, 90 minutes, 2 hours, 3 hours, from 10 minutes to 40 minutes, from 20 minutes to 40 minutes, from 30 minutes to 60 minutes, etc.

In some embodiments, the cap includes a fluid inlet port in addition to the pressure inlet port. In some embodiments, the cap includes a plurality of fluid inlet ports, e.g., from 1 to 3 ports, 1 to 5 ports, 2 to 5 ports, or 1, 2, 3, 4, 5, etc., ports. In some embodiments, the fluid inlet port may be in fluidically communication with a fluid source. In some embodiments, multiple fluids may be added to the reservoir through the same fluid inlet port or different fluid inlet ports. In some embodiments, there may be a fluidic junction before the fluid inlet port, where each branch of the fluidic junction is fluidically connected to a separate fluid source. In some embodiments, the fluid inlet port and the buffer inlet port are the same port. The fluid inlet port may be connected to a fluid source via connection components. In some embodiments, the connection components may be tubes. In some embodiments, the tubing is disposable, allowing sterile delivery of fluid and elution to each reservoir.

Polyribonucleotides may be removed from the filter following pressurization. The polyribonucleotides may be removed through aspiration, e.g., aspirated from a filter surface. In some embodiments, from 1 mg to 1000 mg of circular polyribonucleotides are aspirated from the filter, e.g., from 1 mg to 5 mg, from 1 mg to 10 mg, from 1 mg to 25 mg, from 1 mg to 50 mg, from 1 mg to 75 mg, from 1 mg to 100 mg, from 1 mg to 125 mg, from 1 mg to 150 mg, from 1 mg to 200 mg, from 5 mg to 25 mg, from 5 mg to 50 mg, from 10 mg to 50 mg, from 25 mg to 50 mg, from 25 mg to 50 mg, from 25 mg to 75 mg, from 25 mg to 100 mg, from 25 mg to 125 mg, from 25 mg to 150 mg, from 50 mg to 75 mg, from 50 mg to 100 mg, from 50 mg to 150 mg, from 50 mg to 200 mg, from 50 mg to 250 mg, from 75 mg to 125 mg, from 75 mg to 150 mg, from 75 mg to 200 mg, from 80 mg to 120 mg, from 90 mg to 110 mg, from 100 mg to 150 mg, from 100 mg to 200 mg, from 100 mg to 250 mg, from 125 mg to 175 mg, from 150 mg to 250 mg, from 250 mg to 500 mg, from 250 mg to 750 mg, or from 500 mg to 1000 mg.

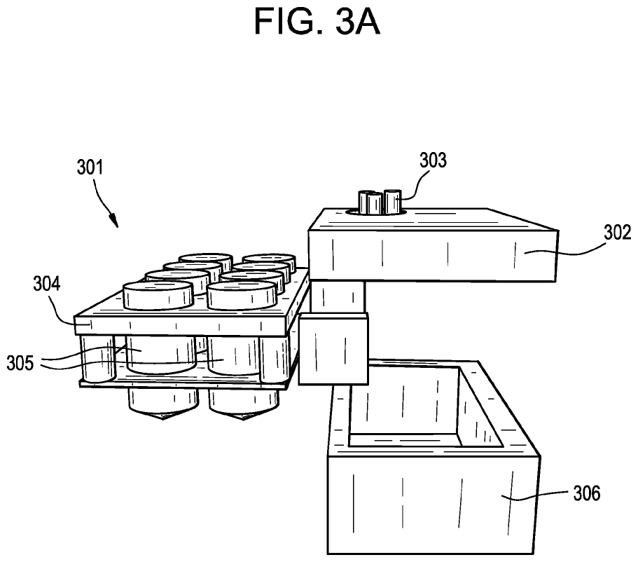

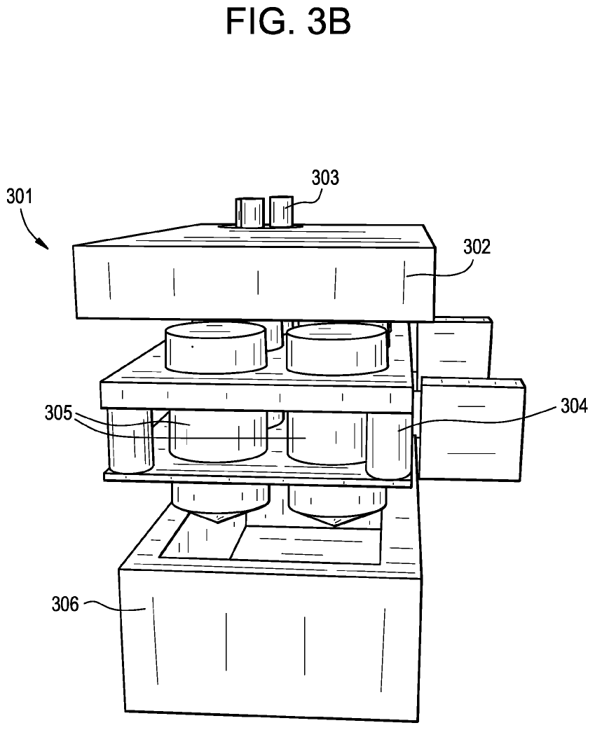

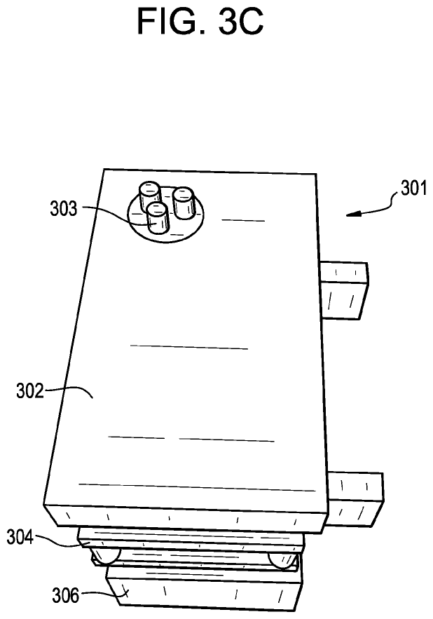

One embodiment of the present invention is a pressurized system (301) or pressurized robot assembly, as seen in FIGS. 3A-3C, including a lid (302) having at least one port (303), a filter tray (304) having a plurality of sample reservoirs (305) having filters, and a waste reservoir (306). In some embodiments, the pressurized system can process a plurality of reservoirs in parallel, such as from 2 reservoirs to 24 reservoirs, preferably from 8 reservoirs to 12 reservoirs, e.g., from 8 reservoirs to 10 reservoirs, 10 reservoirs to 12 reservoirs, or 2, 3, 4, 5, 6, 7, 8, 9, 10, 11, 12, 13, 14, 15, 16, 17, 18, 19, 20, etc., reservoirs. The pressurized system is configured to a variety of reservoir volumes, e.g., both 50 mL and 15 mL reservoirs. In some embodiments, the pressurized system further includes a robotic arm.

In some embodiments, the present devices, systems, and methods may be used to process a plurality of circular polyribonucleotide samples, such as 10 or more, 20 or more, 30 or more, 40 or more, 50 or more, 60 or more, 70 or more, 80 or more, 90 or more, or 100 or more. In some embodiments, a plurality of pressurized systems are used in conjunction, e.g. from 10 to 12 pressurized systems.

In some embodiments, the present devices and systems includes sensors. Sensors may be used to stop the pressurization when the liquid level falls to a certain level in the reservoir. In some embodiments, the liquid detection sensor is selected from the group consisting of an optical sensor, a vibrating sensor, an ultrasonic sensor, a float sensor, a capacitance sensor, a radar sensor, a conductivity sensor, and a resistance sensor. In some embodiments, the sensor is an optical sensor.

In some embodiments, the present devices and systems include at least one safety component or at least one safety feature to avoid operator injury, such as reservoir cracking due to pressurization. For example, high-pressure components, such as reservoirs and connections able to withstand the high pressure, or securement components, such as clamps, may be included.

### Examples

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1

This example demonstrates the use of a pressurization device.

An Amicon® Ultra-15 Filter having a filter porosity of 10 kDa, as shown in FIG. 2, was adapted for buffer exchange using positive pressure. As shown in FIG. 1, a cap was fabricated having a pressure inlet port for use with the Amicon® Ultra-15 Filter. The cap included sealing components in order to aid the system in withstanding higher pressures, for example higher than 15 PSI. A system of components for connection to a positive pressure source was designed for the use of pressurizing the buffer exchange device including the Amicon® Ultra-15 Filter having the retrofitted cap.

For the buffer exchange of circular polyribonucleotides, the cap having the pressure inlet port was first removed, 5 mL of circular polyribonucleotides in solution of an initial buffer 5-10% Acetonitrile in 100 mM TEAA dispensed into the reservoir of the device above the filter, and the cap then replaced. Pressure of 15 PSI was applied to the reservoir through the pressure inlet port using compressed air, such that the first buffer was forced through the filter to the waste collection area, with the circular polyribonucleotides remaining on the filter. The first pressurization took approximately 4 minutes. The compressed air was then turned off and the cap slowly opened. Next, 5 mL of water was dispensed to the reservoir above the filter, the cap replaced, and the pressure reinstated. The process of pausing the pressure, removing the cap, adding the second buffer, replacing the cap and resuming pressure was repeated five times, for a total of six buffer washes with the second buffer. Subsequent pressurized filtrations took between 5 and 40 minutes. The cap was then opened, and 200 μL of circular polyribonucleotides were aspirated from the filter for incorporation into downstream processing.

### Example 2

This example demonstrates the use of a pressurization device.

An Amicon® Ultra-30 Filter Unit having a filter porosity of 10 kDa was adapted for buffer exchange using pressurization as opposed to centrifugation. A cap was fabricated having a pressure inlet port for use with the Amicon® Ultra-30 Filter Unit. The cap included sealing components in order to aid the system in withstanding higher pressures, for example higher than 65 PSI. A system of components for connection to a positive pressure source was designed for the use of pressurizing the device including the Amicon® Ultra-30 Filter Unit having the retrofitted cap.

For the buffer exchange of circular polyribonucleotides, the cap having the pressure inlet port was first removed, 45 mL having 3.1 mg of circular polyribonucleotides in solution of 5-10% Acetonitrile in 100 mM TEAA dispensed into the reservoir of the device above the filter, and the cap then replaced. Pressure of 65 PSI was applied to the reservoir through the pressure inlet port, such that the first buffer was forced through the filter to the waste collection area, with the circular polyribonucleotides remaining on the filter. The first pressurized filtration took approximately 4 minutes. The pressure was then turned off and the cap opened. Next, 10 mL of Sodium Citrate was added to the reservoir above the filter, the cap replaced, and the pressure reinstated. The process of pausing the pressure, removing the cap, adding the second buffer, replacing the cap, and resuming pressure was repeated five times, for a total of six buffer washes with the second buffer. Subsequent pressurized filtrations took between 35 and 50 minutes. The cap was then opened, and 2 mg (1335 ng/μL) of circular polyribonucleotides were aspirated from the filter for incorporation into downstream processing.

### Example 3

This example demonstrates the use of a pressurization device.

An Amicon® Ultra-15 Filter having a filter porosity of 10 kDa was adapted for buffer exchange using pressurization as opposed to centrifugation. As shown in FIG. 1, a cap was fabricated having a pressure inlet port and buffer inlet port for use with the Amicon® Ultra-15 Filter. The cap included sealing components to aid the system in withstanding higher pressures, for example higher than 65 PSI. A system of components for connection to a positive pressure source and a buffer source were designed for the use of pressurizing the buffer exchange device and supplying the buffer exchange with fresh buffer.

For the buffer exchange of circular polyribonucleotides, the cap of the device was first removed, 4 mg of circular polyribonucleotides in 5-10% Acetonitrile in 100 mM TEAA dispensed into the reservoir of the device above the filter, and the cap then replaced. Pressure of 65 PSI was applied to the reservoir through the pressure inlet port, such that the first buffer was forced through the filter to the waste collection area, with the circular polyribonucleotides remaining on the filter. The first pressurized filtration took approximately 12 minutes. The pressure was then paused before 15 mL of sodium citrate added to the reservoir above the filter via the buffer inlet port, and the pressure reinstated. The process of pausing the pressure, adding the second buffer through the buffer inlet port, and resuming pressure was repeated five times, for a total of six buffer washes with the second buffer. Subsequent pressurizations took between 18 and 50 minutes. The cap was then opened, and 3.42 mg (1898.1 ng/μL) circular polyribonucleotides were aspirated from the filter for incorporation into downstream processing.

### Example 4

This example demonstrates the use of an automated pressurization device.

An Amicon® Ultra-15 Filter having a filter porosity of 10 kDa may be adapted for automated buffer exchange using pressurization as opposed to centrifugation. As shown in FIG. 1, a cap was fabricated having a pressure inlet port and buffer inlet port for use with the Amicon® Ultra-15 Filter. The cap included sealing components in order to aid the system in withstanding higher pressures, for example higher than 60 PSI. A system of components for connection to a positive pressure source and a buffer source were designed for the use of pressurizing the buffer exchange device and supplying the buffer exchange with fresh buffer. The device may be further adapted to include a fluid detection sensor.

The buffer exchange may follow the process of Example 1 or Example 2, except that the process is automated through the use of the liquid detection sensor. The liquid detection sensor senses when a determined volume of the first or second buffer or any subsequent has been forced through the filter to the waste collection area. Upon the level of liquid in the reservoir above the filter area reaching a target level, the pressure will be paused, and a predetermined volume of second buffer may be added to the reservoir above the filter. The pressure may then be reinstated. The pausing of the pressure upon a target liquid level being sensed, the addition of second buffer, and the reinstatement of pressure may be repeated five times, for a total of six buffer washes with the second buffer, before the cap is removed and the circular polyribonucleotides on the filter aspirated out for incorporation into either a solution of the second buffer, or into a solution of a third buffer.

1. A method of processing circular polyribonucleotides, the method comprising the steps of:
(a) providing a device comprising: a reservoir comprising a filter disposed between a filtrate chamber and a waste collection chamber; the reservoir further comprising a removable cap having a pressure inlet port; wherein the device is in fluidic communication with a positive pressure source through the pressure inlet port;
(b) providing circular polyribonucleotides in a first fluid in the filtrate chamber;
(c) pressurizing the reservoir with the positive pressure source, wherein the first fluid passes through the filter and the circular polyribonucleotides do not pass through the filter;
(d) applying a second fluid to the reservoir; and
(e) pressurizing the reservoir with the positive pressure source, wherein the second buffer passes through the filter and the circular polyribonucleotides do not pass through the filter.

2. The method of claim 1, further comprising the step of repeating steps (d) and (e) from 1 to 5 times.

3. The method of claim 1 or 2, further comprising the step of (f) aspirating the circular polynucleotides from the filter following step (e).

4. The method of claim 3, wherein 50 mg to 150 mg of circular polyribonucleotides are aspirated from the filter.

5. The method of any one of claims 1-4, wherein the second fluid is applied through a fluid inlet port in the cap.

6. The method of any one of claims 1-5, wherein the fluid inlet port is in fluidic communication with a fluid source.

7. The method of any one of claims 1-6, wherein pressurizing step (c) and (d) comprises pressurizing the reservoir with between 10-100 PSI.

8. The method of any one of claims 1-7, wherein applying step (d) comprises applying from 5 mL to 15 mL of fluid to the reservoir.

9. The method of any one of claims 1-8, wherein the first and/or second fluid is a liquid.

10. The method of claim 9, wherein the liquid is a solution.

11. The method of claim 10, wherein the solution is a buffer.

12. The method of claim 11, wherein the buffer is a formulation buffer, storage buffer, or purification buffer.

13. A device comprising: a reservoir comprising a filtrate chamber and a waste collection chamber separated by a filter; the reservoir further comprising a removable cap in contact with a pressure inlet port; wherein the device is in fluidic communication with a positive pressure source.

14. The device of claim 13, wherein the reservoir is a tube.

15. The device of claim 14, wherein the tube is a centrifugal tube.

16. The device of any one of claims 13-15, wherein the filtrate chamber is funneled.

17. The device of any one of claims 13-16, wherein the device further comprises a sensor.

18. The device of claim 17, wherein the sensor is a liquid detection sensor.

19. The device of any of claim 13-18, wherein the cap further comprises a fluid inlet port.

20. The device of any one of claims 13-19, wherein the volume of the reservoir is from 5 to 100 mL.

21. The device of any one of claims 13-20, wherein the volume of the filtrate chamber is from 5 to 15 mL.

22. The device of any one of claims 13-21, wherein the filter is from 2 to 200 kDa.

23. The device of any one of claims 13-22, wherein the filtrate chamber and filter are stacked on, screwed into, or nested in the reservoir.

24. The device of any one of claims 13-23, wherein the pressure inlet port is sealed.

25. A system comprising a filter tray having a sample reservoir comprising filtrate chamber having a filter, the filter tray disposed between a lid having a pressure inlet port, and a waste reservoir;
wherein the pressure inlet port is in fluidic communication with a positive pressure source.

26. The system of claim 25, wherein the filtrate chamber is funneled.

27. The system of claim 25 or 26, wherein the volume of the filtrate chamber is from 5 mL to 15 mL.

28. The system of any one of claims 25-27, wherein the waste reservoir comprises a gravity or vacuum drain.

29. The system of any one of claims 25-28, wherein the system further comprises a liquid detection sensor.

30. The system of any one of claims 25-29, wherein the system further comprises a vent.

31. The system of any one of claims 25-30, wherein the filter tray, lid, and waste reservoir are sealably connected together.

32. The system of any one of claims 25-31, wherein the filtrate chamber is stacked on, screwed into, or nested in the sample reservoir.

33. The system of any one of claims 25-32, wherein the lid further comprises a fluid inlet port.

34. The system of claim any one of claims 25-33, wherein the filter tray comprises a plurality of sample reservoirs.
